(19) United States
(12) Patent Application Publication
(10) Pub. No.: US 2012/0100135 A1
(43) Pub. Date: Apr. 26, 2012

(54) GENETIC POLYMORPHISMS ASSOCIATED WITH CLINICAL OUTCOMES OF TOPOISOMERASE INHIBITOR THERAPY FOR CANCER

(75) Inventor: Heinz-Josef LENZ, Los Angeles, CA (US)

(73) Assignee: University of Southern California

(21) Appl. No.: 13/278,093

(22) Filed: Oct. 20, 2011

Related U.S. Application Data
(63) Continuation of application No. PCT/US2010/032279, filed on Apr. 23, 2010.
(60) Provisional application No. 61/172,641, filed on Apr. 24, 2009.

(51) Int. Cl. C12Q 1/68; A61P 35/00; A61K 39/395; C40B 30/04; A61K 31/4545

(52) U.S. Cl. 424/133.1; 435/6.11; 506/9; 514/283

(57) ABSTRACT

The invention provides compositions and methods for determining the likelihood of response or survival of cancer patients treated with topoisomerase inhibitor therapy or anti-EGFR and topoisomerase inhibitor therapy combination therapy. After determining if a patient is likely to be successfully treated, the invention also provides methods for treating the patients.

### CROSS REFERENCE TO RELATED APPLICATION

This application claims the benefit under 35 U.S.C. §119(c) of U.S. Provisional Ser. No. 61/172,641, filed Apr. 24, 2009, the contents of which is incorporated by reference in its entirety.

### STATEMENT AS TO FEDERALLY SPONSORED RESEARCH OR DEVELOPMENT

This invention was made with government support under the National Institutes of Health Grant P30 CA 14089. Accordingly, the U.S. Government has certain rights to the invention.

### FIELD OF THE INVENTION

This invention relates to the filed of pharmacogenomics and specifically to the application of genetic polymorphisms to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, sec Lenz (2004) J. Clin. Oncol. 22(13):2519-2521; Park et al. (2006) Cum Opin. Pharma. 6(4):337-344; Zhang et al. (2006) Pharma, and Genomics 16(7):475-483 and U.S. Patent Publ. No. 2006/0115827. For a review of pharmacogenetic and pharmacogenomics in therapeutic antibody development for the treatment of cancer, see Yan and Beckman (2005) Biotechniques 39:565-568.

Although considerable research correlating gene expression and/or polymorphisms has been reported, much work remains to be done. This invention supplements the existing body of knowledge and provides related advantages as well.

### SUMMARY OF THE INVENTION

The invention provides compositions and methods for determining the likelihood of response or survival of cancer patients treated with topoisomerase inhibitor therapy or anti-EGFR and topoisomerase inhibitor therapy combination therapy. After determining if a patient is likely to be successfully treated, the invention also provides methods for treating the patients.

In one aspect, this invention provides a method for identifying a patient having a cancer suitable or not suitable for a topoisomerase inhibitor therapy, comprising, or alternatively consisting essentially of, or yet further consisting of, determining a genotype of a cell or tissue sample isolated from the patient for at least one polymorphism of the group EGFR-CA-repeat in intron 1, MTHFR C677T, or MTHFR A1298C, wherein a genotype of one or more of:

(a) (both alleles with >=20 CA repeats) for EGFR-CA-repeat in intron 1;

(b) (C/C or C/T) for MTHFR C677T; or

(c) (C/C or A/C) for MTHFR A1298C,

identifies the patient as suitable for the topoisomerase inhibitor therapy, or a genotype of one or more of:

(d) (at least one allele with <20 CA repeats) for EGFR-CA-repeat in intron 1;

(e) (T/T) for MTHFR C677T; or

(f) (A/A) for MTHFR A1298C,

identifies the patient as not suitable for the topoisomerase inhibitor therapy. Alternatively, a genotype of none of (a) to (c) identifies the patient as not suitable for the topoisomerase inhibitor therapy.

Also provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for an EGFR-CA-repeat in intron 1 polymorphism, wherein a genotype of (both alleles with >=20 CA repeats) identifies the patient as suitable for the topoisomerase inhibitor therapy, or a genotype of (at least one allele with <20 CA repeats) identifies the patient as not suitable for the topoisomerase inhibitor therapy.

Further provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for a MTHFR C677T polymorphism, wherein a genotype of (C/C or C/T) identifies the patient as suitable for the topoisomerase inhibitor therapy, or a genotype of (T/T) identifies the patient as not suitable for the topoisomerase inhibitor therapy.

Yet further provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for a MTHFR A1298C polymorphism, wherein a genotype of (C/C or A/C) identifies the patient as suitable for the topoisomerase inhibitor therapy, or a genotype of (A/A) identifies the patient as not suitable for the topoisomerase inhibitor therapy.

In one aspect of any of the above noted methods, a patient of a genotype of a group that is suitable for the therapy is a patient that has relatively longer overall survival or progression free survival than patients not having a genotype of the group and having the cancer and receiving the therapy.

In some embodiments, suitability of the patient for the topoisomerase inhibitor therapy is measured clinically. In one aspect, suitability is measured by the patient's progression free survival. In another aspect, suitability of the patient for the topoisomerase inhibitor therapy is measured by the patient's overall survival.

This invention provides a method for identifying a patient having a cancer more or less likely to respond a topoisomerase inhibitor therapy, comprising, or alternatively consisting essentially of, or yet further consisting of, determining a genotype of a cell or tissue sample isolated from the patient for a MTHFR A1298C polymorphism, wherein a genotype of (C/C or A/A) for MTHFR A1298C identifies the patient as more likely to respond to the topoisomerase inhibitor therapy, or a genotype of (A/C) for MTHFR A1298C identifies the patient as less likely to respond to the topoisomerase inhibitor therapy. In one aspect, a genotype of (C/C or A/A) for MTHFR A1298C identifies the patient as more likely to respond to the topoisomerase inhibitor therapy. In another aspect, a genotype of (A/C) for MTHFR A1298C identifies the patient as less likely to respond to the topoisomerase inhibitor therapy.

In one aspect, a patient that is more likely to respond to the topoisomerase therapy is a patient that is relatively more likely to respond to the topoisomerase therapy than patients having a genotype of (A/C) for MTHFR A1298C and having the cancer and receiving the therapy.

This invention also provides use of a topoisomerase inhibitor therapy for the therapy of a patient selected for suitable for the therapy based on any of the above noted methods.

Thus, this invention also provides a method for treating a cancer patient is provided, the method comprising, or alternatively consisting essentially of, or yet further consisting of,

(a) identifying a cancer patient suitable for a topoisomerase inhibitor therapy by determining a cell or tissue sample isolated from the patient to have a genotype of at least one of i) (both alleles with >=20 CA repeats) for EGFR-CA-repeat in intron 1, ii) (C/C or C/T) for MTHFR C677T, or iii) (C/C or A/C) for MTHFR A1298C; and
(b) administering to the patient an effective amount of the topoisomerase therapy, thereby treating the patient.

Further provided is a method for treating a cancer patient, comprising, or alternatively consisting essentially of, or yet further consisting of administering to the patient a topoisomerase therapy, wherein the patient is selected for the therapy based on a genotype of at least one of i) (both alleles with >=20 CA repeats) for EGFR-CA-repeat in intron 1, ii) (C/C or C/T) for MTHFR C677T, or iii) (C/C or A/C) for MTHFR A1298C, in a sample isolated from the patient, thereby treating the patient.

Yet further provided is a method for treating a patient having a cancer, comprising, or alternatively consisting essentially of, or yet further consisting of, administering a topoisomerase therapy, wherein the patient is selected for the therapy based on a genotype of (both alleles with >=20 CA repeats) for the EGFR-CA-repeat in intron 1 polymorphism in a sample isolated from the patient, thereby treating the patient.

Also provided is a method for treating a patient having a cancer, comprising, or alternatively consisting essentially of, or yet further consisting of, administering a topoisomerase therapy, wherein the patient is selected for the therapy based on a genotype of (C/C or C/T) for the MTHFR C677T polymorphism, in a sample isolated from the patient, thereby treating the patient.

Yet further provided is a method for treating a patient having a cancer, comprising, or alternatively consisting essentially of, or yet further consisting of, administering a topoisomerase therapy to a patient selected for the therapy based on a genotype of (C/C or A/C) for the MTHFR A1298C polymorphism, thereby treating the patient.

In one aspect of any of the above noted methods, a patient of a genotype of a group that is selected for the therapy or suitable for the therapy is a patient that has relatively longer overall survival or progression free survival than patients not having a genotype of the group and having the cancer and receiving the therapy.

In some embodiments, suitability or selection of the patient for the topoisomerase inhibitor therapy is measured clinically. In one aspect, suitability is measured by the patient's progression free survival. In another aspect, suitability of the patient for the topoisomerase inhibitor therapy is measured by the patient's overall survival.

In one aspect, a patient that is more likely to respond to the topoisomerase therapy or selected for the therapy and therefore treated is a patient that is relatively more likely to respond to the topoisomerase therapy than patients having a genotype of (A/C) for MTHFR A1298C and having the cancer and receiving the therapy.

Also provided is a kit for use in identifying a cancer patient suitable for a topoisomerase inhibitor therapy, comprising, or alternatively consisting essentially of, or yet further consisting of, one or more of the group of suitable primers or probes or a microarray for screening at least one polymorphism of the group EGFR-CA-repeat in intron 1, MTHFR C677T, or MTHFR A1298C, and instructions for use therein.

In one aspect of any of the above noted methods, the topoisomerase inhibitor therapy comprises, or alternatively consists essentially of, or yet further consists of, administration of irinotecan or an equivalent thereof. In some embodiments, the topoisomerase inhibitor therapy comprises, or alternatively consists essentially of, or yet further consists of, administration of a topoisomerase inhibitor in combination with an anti-EGFR drug, in a particular aspect, the anti-EGFR drug is cetuximab or an equivalent thereof. The administration of the topoisomerase inhibitor and the anti-EGFR drug can be concurrent or sequential.

In some embodiments of the invention, the topoisomerase inhibitor therapy is a second line therapy.

In the above noted methods, use or kit, the cancer patient is suffering from at least one cancer of the type of the group: head and neck cancer or colorectal cancer. In one aspect, the cancer patient is suffering at least colorectal. In a particular aspect, the colorectal cancer is metastatic colorectal cancer.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS U. H. Miller and M. P. Cabs eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR′S HANDBOOK OF EXPERIMENTAL IMMUNOLOGY (L. A. Herzenberg et al. eds (1996)).

### DEFINITIONS

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

An anti-EGFR therapy refers to an antibody or a small molecule inhibiting expression or function of EGFR. In one aspect, anti-EGFR therapy comprises, or alternatively consists essentially of, or yet further, consists of an antibody or fragment thereof that binds the EGFR antigen. A non-limiting example of such is the antibody cetuximab or equivalents thereof that bind to the same epitope. It can be polyclonal or monoclonal. The antibody may be of any appropriate species such as for example, murine, ovine or human. It can be humanized, chimeric, recombinant, bispecific, a heteroantibody, a derivative or variant of a polyclonal or monoclonal.

Cetuximab (IMC-C225) is marketed under the name Erbitux®. Cetuximab is a chimeric (mouse/human) monoclonal antibody, an epidermal growth factor receptor (EGFR) inhibitor, given by intravenous injection for treatment of metastatic colorectal cancer and head and neck cancer. Cetuximab is manufactured and distributed in North America by ImClone and Bristol-Myers Squibb, while in the rest of the world distribution is by Merck KGaA. In one aspect, an equivalent of cetuximab is an antibody directed to EGFR, or an antibody that binds to the same epitope as cetuximab, or a small molecule targeting EGFR or inhibiting EGFR. In another aspect, an equivalent of cetuximab may also includes homologs of cetuximab, mutant cetuximab, recombinant cetuximab that retains substantially the same function of cetuximab.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website as www.cancer.gov, last visited on May 1, 2008. Typically, a patient is given a subsequent chemotherapy regimen because the patient did not shown a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody. An example of an equivalent cetuximab antibody is one which binds to and inhibits the biologic activity of human epidermal growth factor receptor (EGFR).

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides, e.g., A/C for the MTHFR gene at position +1298. If the allelic composition of a SNP locus is heterozygous, the genotype of the SNP locus will be identified as “X/X” wherein X identifies the nucleotide that is present at both alleles, e.g., A/A for MTHFR gene at position +1298. In another aspect, a polymorphic locus harbors allelic variants of nucleotide sequences of different length. The genotype of the polymorphic locus will be identified with the length of the allelic variant, e.g., at least one allele with <20 CA repeats at intron 1 of the EGFR gene. The genotype of the cell or tissue sample will be identified as a combination of genotypes of all polymorphic loci of interest, e.g. A/A for MTHFR gene at position +1298 and both alleles with <20 CA repeats at intron 1 of the EGFR gene.

The term “genetic marker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

The term “genotype” refers to the specific allelic composition of an entire cell or a certain gene and in some aspects a specific polymorphism associated with that gene, whereas the term “phenotype” refers to the detectable outward manifestations of a specific genotype.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more desirable clinical outcome as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, amore desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, amore desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same disease and receiving the same therapy but not possessing the characteristic. As defined herein, the patients is considered suitable for the therapy. In another such aspect, a patient possessing a characteristic may exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “Stage I cancer,” “Stage II cancer,” “Stage III cancer,” and “Stage IV” refer to the TNM staging classification for cancer. Stage I cancer typically identities that the primary tumor is limited to the organ of origin. Stage II intends that the primary tumor has spread into surrounding tissue and lymph nodes immediately draining the area of the tumor, Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

“Having the same cancer” is used when comparing one patient to another or alternatively, one patient population to another patient population. For example, the two patients or patient population will each have or be suffering from colon cancer.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

As used herein, the term “patient” intends an animal, a mammal or yet further a human patient. For the purpose of illustration only, a mammal includes but is not limited to a human, a simian, a murine, a bovine, an equine, a porcine or an ovine.

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

If an antibody is used in combination with the above-noted chemotherapy or for diagnosis or as an alternative to the chemotherapy, the antibodies can be polyclonal or monoclonal and can be isolated from any suitable biological source, e.g., murine, rat, sheep and canine. Additional sources are identified infra.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)₂ fragment, a bivalent fragment comprising two Fab fragments linked by a disulfide bridge at the hinge region; a Fd fragment consisting of the VH and CH, domains; Fv fragment consisting of the VL and VH domains of a single arm of an antibody, a dAb fragment (Ward et al. (1989) Nature 341:544-546), which consists of a VU domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “antibody variant” is intended to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by heavy chain constant region genes.

### DESCRIPTIVE EMBODIMENTS

The invention further provides diagnostic, prognostic and therapeutic methods, which are based, at least in part, on determination of the identify of a genotype of interest identified herein.

For example, information obtained using the diagnostic assays described herein is useful for determining if a subject is suitable for cancer treatment of a given type or is likely to experience tumor recurrence. Based on the prognostic information, a doctor can recommend a therapeutic protocol, useful for reducing the malignant mass or tumor in the patient or treat cancer in the individual.

Determining, whether a subject as more or less likely to experience a relatively longer overall survival or progression free survival than patients not having a genotype of the group and having the cancer and receiving the therapy.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods of this invention are useful for the diagnosis, prognosis and treatment of patients suffering from at least one or more cancer of the group: metastatic or non-metastatic rectal cancer, metastatic or non-metastatic colon cancer, metastatic or non-metastatic colorectal cancer, lung cancer, head and neck cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, colorectal cancer or head and neck cancer. In one aspect, the colorectal cancer is metastatic colorectal cancer. In a further aspect, the patients

The methods are useful in the assistance of an animal, a mammal or yet further a human patient. For the purpose of illustration only, a mammal includes but is not limited to a human, a simian, a murine, a bovine, an equine, a porcine or an ovine.

### Diagnostic Methods

This invention provides a method for identifying a patient having a cancer suitable or not suitable for a topoisomerase inhibitor therapy, comprising, or alternatively consisting essentially of, or yet further consisting of, determining a genotype of a cell or tissue sample isolated from the patient for at least one polymorphism of the group EGFR-CA-repeat in intron 1, MTHFR C677T, or MTHFR A1298C, wherein a genotype of one or more of:

(a) (both alleles with >=20 CA repeats) for EGFR-CA-repeat in intron 1;

(b) (C/C or C/T) for MTHFR C677T; or

(c) (C/C or A/C) for MTHFR A1298C,

identifies the patient as suitable for the topoisomerase inhibitor therapy, or a genotype of none of the above or one or more of:

(d) (at least one allele with <20 CA repeats) EGFR-CA-repeat in intron 1;

(e) (T/T) for MTHFR C677T; or

(f) (A/A) for MTHFR A1298C,

identifies the patient as not suitable for the topoisomerase inhibitor therapy. Alternatively, a genotype of none of (a) to (c) identifies the patient as not suitable for the topoisomerase inhibitor therapy.

In one aspect, a genotype of one or more of:

(a) (both alleles with 20 CA repeats) for EGFR-CA-repeat in intron 1;

(b) (C/C or C/T) for MTHFR C677T; or

(c) (C/C or A/C) for MTHFR A1298C,

identifies the patient as suitable for the topoisomerase inhibitor therapy.
In another aspect, a genotype of one or more of:

(d) (at least one allele with <20 CA repeats) for EGFR-CA-repeat in intron I;

(e) (T/T) for MTHFR C677T; or

(f) (A/A) for MTHFR A1298C,

identifies the patient as not suitable for the topoisomerase inhibitor therapy.

Also provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for an EGFR-CA-repeat in intron 1 polymorphism, wherein a genotype of (both alleles with >=20 CA repeats) identifies the patient as suitable for the topoisomerase inhibitor therapy, or a genotype of (at least one allele with <20 CA repeats) identifies the patient as not suitable for the topoisomerase inhibitor therapy.

Further provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for a MTHFR C677T polymorphism, wherein a genotype of (C/C or C/T) identifies the patient as suitable for the topoisomerase inhibitor therapy, or a genotype of (T/T) identifies the patient as not suitable for the topoisomerase inhibitor therapy.

Yet further provided is a method for identifying a patient having a cancer suitable or not suitable for a topoisomerase inhibitor therapy, comprising, or alternatively consisting essentially of, or yet further consisting of, determining a genotype of a cell or tissue sample isolated from the patient for a MTHFR A1298C polymorphism, wherein a genotype of (C/C or A/C) identifies the patient as suitable for the topoisomerase inhibitor therapy, or a genotype of (A/A) identifies the patient as not suitable for the topoisomerase inhibitor therapy.

In one aspect of any of the above noted methods, a patient of a genotype of a group that is suitable for the therapy is a patient that has relatively longer overall survival or progression free survival than patients not having a genotype of the group and having the cancer and receiving the therapy.

In some embodiments, suitability of the patient for the topoisomerase inhibitor therapy is measured clinically. In one aspect, suitability is measured by the patient's progression free survival. In another aspect, suitability of the patient for the topoisomerase inhibitor therapy is measured by the patient's overall survival.

This invention also provides a method for identifying a patient having a cancer more or less likely to respond a topoisomerase inhibitor therapy, comprising, or alternatively consisting essentially of, or yet further consisting of, determining a genotype of a cell or tissue sample isolated from the patient for a MTHFR A1298C polymorphism, wherein a genotype of (C/C or A/A) for MTHFR A1298C identifies the patient as more likely to respond to the topoisomerase inhibitor therapy, or a genotype of (A/C) for MTHFR A1298C identifies the patient as less likely to respond to the topoisomerase inhibitor therapy. In one aspect, a genotype of (C/C or A/A) for MTHFR A1298C identifies the patient as more likely to respond to the topoisomerase inhibitor therapy. In another aspect, a genotype of (A/C) for MTHFR A1298C identifies the patient as less likely to respond to the topoisomerase inhibitor therapy.

In one aspect, a patient that is more likely to respond to the topoisomerase therapy is a patient that is relatively more likely to respond to the topoisomerase therapy than patients having a genotype of (A/C) for MTHFR A1298C and having the cancer and receiving the therapy.

Also provided is a method for treating a cancer patient, comprising, or alternatively consisting essentially of, or yet further consisting of:

(a) determining a genotype of a cell or tissue sample isolated from the patient for at least one polymorphism of the group i) EGFR-CA-repeat in intron 1, ii) MTHFR C677T, or iii) MTHFR A1298C;
(b) identifying the patient having a genotype of one or more of i) (both alleles with >=20 CA repeats) for EGFR-CA-repeat in intron 1, ii) (C/C or C/T) for MTHFR C677T, or iii) (C/C or A/C) for MTHFR A1298C; and
(c) subjecting the patient identified in step (b) to a topoisomerase inhibitor therapy, thereby treating the patient.

This invention also provides use of a topoisomerase inhibitor therapy for the therapy of a patient selected for suitable for the therapy based on any of the above noted methods.

In one aspect of any of the above noted methods, the topoisomerase inhibitor therapy comprises, or alternatively consists essentially of, or yet further consists of, administration of irinotecan or an equivalent thereof. In some embodiments, the topoisomerase inhibitor therapy comprises, or alternatively consists essentially of, or yet further consists of, administration of a topoisomerase inhibitor in combination with an anti-EGFR drug. In a particular aspect, the anti-EGFR drug is cetuximab or an equivalent thereof. The administration of the topoisomerase inhibitor and the anti-EGFR drug can be concurrent or sequential.

In some embodiments of the invention, the topoisomerase inhibitor therapy is a second line therapy.

in the above methods, the cancer patient is suffering from at least one cancer of the type of the group: head and neck cancer, metastatic or non-metastatic rectal cancer, metastatic or non-metastatic colon cancer, metastatic or non-metastatic colorectal cancer, non-small cell lung cancer, metastatic breast cancer, non-metastatic breast cancer, renal cell carcinoma, glioblastoma multiforme, ovarian cancer, hormone-refractory prostate cancer, non-metastatic unresectable liver cancer, or metastatic or unresectable locally advanced pancreatic cancer. In one aspect, the cancer patient is suffering from colorectal cancer. In a yet further aspect, the cancer patient is suffering from metastatic colorectal cancer.

Suitable patient samples in the methods include, but are not limited to a sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. The samples can be at least one of a fixed tissue, a frozen tissue, a biopsy tissue, a resection tissue, a microdissected tissue, or combinations thereof.

Methods to determine the genotype of the patient sample can comprise, or alternatively consisting essentially of, or yet further consist of PCR, PCR-RFLP, sequencing, or microarray.

The methods are useful in the assistance of an animal, a mammal or yet further a human patient. For the purpose of illustration only, a mammal includes but is not limited to a simian, a murine, a bovine, an equine, a porcine or an ovine.

Any suitable method for identifying the genotype in the patient sample can be used and the inventions described herein are not to be limited to these methods. For the purpose of illustration only, the genotype is determined by a method comprising, or alternatively consisting essentially of, or yet further consisting of, hybridization, PCR or more specifically, PCR-RFLP.

### Polymorphic Region

For example, information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci. USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is delectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors,

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site, Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. I N. et al. (199)) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

lithe polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support is used as a support capable of binding of a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. Nos. 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. Nos. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. Nos. 5,576,180; 6,106,777 or 7,179,600) A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. Nos. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. Nos. 6,811,982; 6,835,541 or 7,125,673). DNA is first treated with sodium bisulfite, which converts unmethylated cytosine bases to uracil, which is recognized by PCR primers as thymine, Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. Nos. 5,882,856; 6,531,282 or 7,118,867). By targeting multiple genes at once, additional information may be gained from a single test run that otherwise would require several times the reagents and more time to perform. Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. Nos. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. Nos. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci. USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. Nos. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TA IL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₘ of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S, and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genetic polymorphisms identified herein. The prognostic panel comprises probes or primers that can be used to amplify and/or for determining the molecular structure of the polymorphisms identified herein. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfiuidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the TS gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucicotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Methods of Treatment

The invention further provides methods of treating patients having solid malignant tissue mass or tumor from the group head and neck cancer, metastatic or non-metastatic rectal cancer, metastatic or non-metastatic colon cancer, metastatic or non-metastatic colorectal cancer, non-small cell lung cancer, metastatic breast cancer, non-metastatic breast cancer, head and neck cancer, renal cell carcinoma, glioblastoma multiforme, ovarian cancer, hormone-refractory prostate cancer, non-metastatic unresectable liver cancer, or metastatic or unresectable locally advanced pancreatic cancer.

The treatment comprises, or alternatively consists essentially of, or yet further consists of (a) determining a genotype of a cell or tissue sample isolated from the patient for at least one polymorphism of the group i) EGFR-CA-repeat in intron 1, ii) MTHFR C677T, or iii) MTHFR A1298C;

(b) identifying the patient having a genotype of one or more of i) (both alleles with >=20 CA repeats) for EGFR-CA-repeat in intron 1, ii) (C/C or C/T) for MTHFR C677T, or (C/C or A/C) for MTHFR A1298C; and
(c) subjecting the patient identified in step (b) to a topoisomerase inhibitor therapy, thereby treating the patient.

Further provided is a method for treating a cancer patient, comprising, or alternatively consisting essentially of, or yet further consisting of administering to the patient a topoisomerase therapy, wherein the patient is selected for the therapy based on a genotype of at least one of i) (both alleles with >=20 CA repeats) for EGFR-CA-repeat in intron 1, ii) (C/C or C/T) for MTHFR C677T, or iii) (C/C or A/C) for MTHFR A1298C, in a sample isolated from the patient, thereby treating the patient.

Yet further provided is a method for treating a patient having a cancer, comprising, or alternatively consisting essentially of, or yet further consisting of, administering a topoisomerase therapy, wherein the patient is selected for the therapy based on a genotype of (both alleles with >=20 CA repeats) for the EGFR-CA-repeat in intron 1 polymorphism in a sample isolated from the patient, thereby treating the patient.

Also provided is a method for treating a patient having a cancer, comprising, or alternatively consisting essentially of, or yet further consisting of, administering a topoisomerase therapy, wherein the patient is selected for the therapy based on a genotype of (C/C or C/T) for the MTHFR C677T polymorphism, in a sample isolated from the patient, thereby treating the patient.

Yet further provided is a method for treating a patient having a cancer, comprising, or alternatively consisting essentially of, or yet further consisting of, administering a topoisomerase therapy to a patient selected for the therapy based on a genotype of (C/C or A/C) for the MTHFR A1298C polymorphism, thereby treating the patient.

In one aspect of any of the above noted methods, a patient of a genotype of a group that is selected for the therapy or suitable for the therapy is a patient that has relatively longer overall survival or progression free survival than patients not having a genotype of the group and having the cancer and receiving the therapy.

In some embodiments, suitability or selection of the patient for the topoisomerase inhibitor therapy is measured clinically. In one aspect, suitability is measured by the patient's progression free survival. In another aspect, suitability of the patient for the topoisomerase inhibitor therapy is measured by the patient's overall survival.

In one aspect, a patient that is more likely to respond to the topoisomerase therapy or selected for the therapy and therefore treated is a patient that is relatively more likely to respond to the topoisomerase therapy than patients having a genotype of (A/C) for MTHFR A1298C and having the cancer and receiving the therapy.

Also provided is a kit for use in identifying a cancer patient suitable for a topoisomerase inhibitor therapy, comprising, or alternatively consisting essentially of, or yet further consisting of, one or more of the group of suitable primers or probes or a microarray for screening at least one polymorphism of the group EGFR-CA-repeat in intron I, MTHFR C677T, or MTHFR A1298C, and instructions for use therein.

In one aspect of any of the above noted methods, the topoisomerase inhibitor therapy comprises, or alternatively consists essentially of, or yet further consists of, administration of irinotecan or an equivalent thereof. In some embodiments, the topoisomerase inhibitor therapy comprises, or alternatively consists essentially of, or yet further consists of, administration of a topoisomerase inhibitor in combination with an anti-EGFR drug. In a particular aspect, the anti-EGFR drug is cetuximab or an equivalent thereof. The administration of the topoisomerase inhibitor and the anti-EGFR drug can be concurrent or sequential.

In some embodiments of the invention, the topoisomerase inhibitor therapy is a second line therapy.

Suitable samples for the methods as described herein, the sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. The samples can be any one or more of a fixed tissue, a frozen tissue, a biopsy tissue, a resection tissue, a microdissected tissue, or combinations thereof.

Accordingly, a formulation comprising the necessary chemotherapy or chemical equivalent thereof is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

The chemotherapeutic agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Martin REMINGTON′S PHARM. SCI., 15th Ed. (Mack Publ. Co., Easton (1975) and Williams & Williams, (1995), and in the “PHYSICIAN′S DESK REFERENCE”, 52^{nd} ed., Medical Economics, Montvale, N.J. (1998).

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-lcucovorin, fluorouracil/oxaliplatin, methotrexate-leucovorin, and the like.

Combinations of chemotherapies and molecular targeted therapies, biologic therapies, and radiation therapies are also well known to the art; including therapies such as trastuzumab plus paclitaxel, alone or in further combination with platinum compounds such as oxaliplatin, for certain breast cancers, and many other such regimens for other cancers; and the “Dublin regimen” 5-fluorouracil IV over 16 hours on days 1-5 and 75 mg/m² cisplatin IV or oxaliplatin over 8 hours on day 7, with repetition at 6 weeks, in combination with 40 Gy radiotherapy in 15 fractions over the first 3 weeks) and the “Michigan regimen” (fluorouracil plus cisplatin or oxaliplatin plus vinblastine plus radiotherapy), both for esophageal cancer, and many other such regimens for other cancers, including colorectal cancer.

In another aspect of the invention, the method for treating a patient further comprises, or alternatively consists essentially of, or yet further consists of surgical resection of a metastatic or non-metastatic solid malignant tumor and, in some aspects, in combination with radiation. Methods for treating these tumors as Stage I, Stage II, Stage III, or Stage IV by surgical resection and/or radiation are known to one skilled in the art. Guidelines describing methods for treatment by surgical resection and/or radiation can be found at the National Comprehensive Cancer Network's web site, nccn.org, last accessed on May 27, 2008.

The invention provides an article of manufacture, comprising packaging material and at least one vial comprising a solution of the chemotherapy as described herein and/or or at least one antibody or its biological equivalent with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising the chemotherapy and/or at least one lyophilized antibody or its biological equivalent and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

Chemotherapeutic formulations of the present invention can be prepared by a process which comprises mixing at least one antibody or biological equivalent and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing of the antibody and preservative in an aqueous diluent is carried out using conventional dissolution and mixing procedures. For example, a measured amount of at least one antibody in buffered solution is combined with the desired preservative in a buffered solution in quantities sufficient to provide the antibody and preservative at the desired concentrations. Variations of this process would be recognized by one of skill in the art, e.g., the order the components are added, whether additional additives are used, the temperature and pH at which the formulation is prepared, are all factors that can be optimized for the concentration and means of administration used.

The compositions and formulations can be provided to patients as clear solutions or as dual vials comprising a vial of lyophilized antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available. Recognized devices comprising these single vial systems include those pen-injector devices for delivery of a solution such as BD Pens, BD Autojectore, Humaject® NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J. available at bectondickenson.com), Disetronic (Burgdorf, Switzerland, available at disetronic.com; Bioject, Portland, Oreg. (available at bioject.com); National Medical Products, Weston Medical (Peterborough, UK, available at weston-medical.com), Medi-Ject Corp (Minneapolis, Minn., available at mediject.com).

Various delivery systems are known and can be used to administer a chemotherapeutic agent of the invention, e.g., encapsulation in liposomes, microparticles, microcapsules, expression by recombinant cells, receptor-mediated endocytosis. Sec e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the invention locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Also provided is a therapy or a medicament comprising an effective amount of a chemotherapeutic as described herein for treatment of a human cancer patient having the polymorphism of the gene of interest as identified in the experimental examples.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the cancer being treated and the patient, and the subject being treated. Single or multiple administrations can be carried out with the dose level and pattern being selected by the treating physician.

### Kits

As set forth herein, the invention provides diagnostic methods for determining the polymorphic region of the gene of interest. In some embodiments, the methods use probes or primers or a microarray comprising nucleotide sequences which are complementary to the gene of interest. Accordingly, the invention provides kits for performing these methods as well as instructions for carrying out the methods of this invention such as collecting tissue and/or performing the screen, and/or analyzing the results, and/or administration of an effective amount of a 5-FU based adjuvant therapy or equivalents of each thereof. These can be used alone or in combination with other suitable chemotherapy or biological therapy. In an alternative aspect, the kit further comprises, or alternatively consists essentially of, or yet further consists of, a 5-FU based adjuvant therapy as defined herein and optionally instructions for use of the therapy to treat the cancer patient.

In an embodiment, the invention provides a kit for determining whether a subject is likely responsive to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene. Oligonucleotides “specific for” the gene of interest bind either to the gene of interest or bind adjacent to the gene of interest. For oligonucleotides that are to be used as primers for amplification, primers are adjacent if they are sufficiently close to be used to produce a polynucleotide comprising the gene of interest. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

In one aspect, provided is a kit for use in identifying a cancer patient suitable Dora topoisomerase inhibitor therapy, comprising, or alternatively consisting essentially of, or yet further consisting of, suitable primers or probes for screening at least one polymorphism of the group EGFR-CA-repeat in intron 1, MTHFR C677T, or MTHFR A1298C, and instructions for use therein.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S. and Thompson, eds., (1991) GENETICS IN MEDICINE, W B Saunders Co., Philadelphia, Pa. This is useful, e.g., in forensic studies.

The invention now being generally described, it will be more readily understood by reference to the following example which is included merely for purposes of illustration of certain aspects and embodiments of the present invention, and are not intended to limit the invention.

### EXPERIMENTAL DETAILS

### Example 1

Background: EPIC, a multinational phase III clinical trial with IR+CB vs IR alone in mCRC patients in the second-line setting after failure of FOLFOX demonstrated a benefit for IR+CB in progression-free survival (PFS) and response rate (RR). This study was to test functional germline polymorphisms involved in the EGFR—(EGF, EGFR), angiogenesis—(VEGF, IL-8, CXCR-2)—and drug-metabolism related genes (UGT1A1, MTHFR) for their potential role as molecular predictors for clinical outcome in patients treated with CB/IR vs. IR alone.

Material and Methods: DNA was extracted from all available formalin-fixed paraffin-embedded tumor samples from the phase III EPIC trial (US sites only). Genotyping was performed using PCR-RFLP assays and 5′-end [g-33P] ATP′ labeled PCR-protocols.

TABLE 1
Primer Sequences and Annealing Temperatures for Determining Polymorphisms

| | | | Temp. |
|---|---|---|---|
| Polymorphism | Forward Primer | Reverse Primer | (° C.) |
| EGFR-CA-repeat | ACCCCAGGGCTCTATGGGAA | TGAGGGCACAAGAAGCCCCT | 55 |
| in intron 1 | (SEQ ID NO. 1) | (SEQ ID NO. 2) | |
| MTHFR 677 | TGAAGGAGAAGGTGTCTGCGGGA | AGGACGGTGCGGTGAGAGTG | |
| | (SEQ ID NO. 3) | (SEQ ID NO. 4) | |
| MTHFR 1298 | CTTTGGGGAGCTGAAGGACTACTAC | CACTTTGTGACCATTCCGGTTTG | |
| | (SEQ ID NO. 5) | (SEQ ID NO. 6) | |

Results: 186 patients were treated either with IR/CB (arm A, 84 patients) or IR (arm B, 102 patients) only. In arm A, 11/84 patients (13%) showed CR or PR, whereas 73/84 (87%) patients had SD or PD. For arm B, 6/102 patients (6%) showed CR or PR, whereas 96/102 patients (94%) had SD or PD. Median PFS in arm A was 3.0 months (95% CI: 2.4-4.1 months) vs 2.7 months (95% CI: 2.2-2.9 months) in arm B; median overall survival (OS) was 9.3 months (95% CI: 7.1-12.1 months) in arm A vs. 12.3 months (95% CI: 10.4-17.9 months) in arm B. K-ras mutation status was not significantly associated with PFS or response to CB/IR in the subgroup of 186 patients. It was found an EGFR-CA-repeat in intron I in arm A was associated with PFS (p=0.031, log-rank test) (Table 2). In arm A, a significant association with relative risk (RR) (p=0.0103, Fisher's exact test) for MTHFR 1298 was found (Table 3). MTHFR 677 (p=0.0048, log-rank test) and MTHFR 1298 (p=0.038, log-rank test) were found to be associated with OS in arm B (Table 4). In multivariate analysis, EGFR-CA-repeat was significantly associated with PFS (adjusted p=0.023). Furthermore, MTHFR 677 and MTHFR 1298 was associated with OS (adjusted p=0.028 and 0.026, respectively, Cox-proportional hazards models), independent from K-ras mutation status, race and number of disease sites.

TABLE 2
polymorphism associated with progress free survival in arm A.

| | Longer PFS | Shorter PFS |
|---|---|---|
| EGFR-CA-repeat intron 1 | Both alleles | At least 1 allele |
| (CA repeat 14-26) | with >=20 CA repeats | with <20 CA repeats |

TABLE 3
polymorphism associated with relative risk or response in arm A.

| | Higher response | Lower response |
|---|---|---|
| MTHFR A1298C | A/A or C/C (20%) | A/C (0%) |

TABLE 4
polymorphism associated with overall survival in arm B.

| | Longer OS | Shorter OS |
|---|---|---|
| MTHFR A1298C | C/C or A/C | A/A |
| MTHFR C677T | C/C or C/T | T/T |

Conclusions: This study demonstrates the potential predictive value of polymorphisms in the EGFR- and MTHFR-gene in mCRC patients treated with IR+CB.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying a cancer patient suitable or not suitable for a therapy comprising a topoisomerase inhibitor, comprising determining a genotype of a cell or tissue sample isolated from the patient for at least one polymorphism of the group EGFR-CA-repeat in intron 1, MTHFR C677T, or MTHFR A1298C, wherein the presence of the genotype of one or more of:
(a) (both alleles with >=20 CA repeats (SEQ ID NO: 7)) for EGFR-CA-repeat in intron 1;
(b) (C/C or C/T) for MTHFR C677T; or
(c) (C/C or A/C) for MTHFR A1298C,
identifies the patient as suitable for the topoisomerase inhibitor therapy, or the presence of none of (a) to (c) identifies the patient as not suitable for the topoisomerase inhibitor therapy.

2. The method of claim 1, wherein the presence of the genotype of one or more of:
(a) (both alleles with >=20 CA repeats (SEQ ID NO: 7)) for EGFR-CA-repeat in intron 1;
(b) (C/C or C/T) for MTHFR C677T; or
(c) (C/C or A/C) for MTHFR A1298C,
identifies the patient as suitable for the topoisomerase inhibitor therapy.

3. The method of claim 1, wherein the presence of none of (a) to (c) identifies the patient as not suitable for the topoisomerase inhibitor therapy.

4. A method for selecting or not selecting a cancer patient for a therapy comprising a topoisomerase inhibitor, comprising determining a genotype of a cell or tissue sample isolated from the patient for at least one polymorphism of the group EGFR-CA-repeat in intron 1, MTHFR C677T, or MTHFR A1298C, wherein the patient is selected for the therapy if the genotype of one or more of:
(a) (both alleles with >=20 CA repeats (SEQ ID NO: 7)) for EGFR-CA-repeat in intron 1;
(b) (C/C or C/T) for MTHFR C677T; or
(c) (C/C or A/C) for MTHFR A1298C,
is present, or the patient is not selected for the therapy if none of (a) to (c) is present.

5. The method of claim 4, wherein the patient is selected for the therapy if the genotype of one or more of:
(a) (both alleles with >=20 CA repeats (SEQ ID NO: 7)) for EGFR-CA-repeat in intron 1;
(b) (C/C or C/T) for MTHFR C677T; or
(c) (C/C or A/C) for MTHFR A1298C,
is present.

6. The method of claim 4, wherein the patient is not selected for the therapy if none of (a) to (c) is present.

7. A method for treating a cancer patient selected for a therapy comprising an topoisomerase inhibitor based on the presence of a genotype of one or more of:
(a) (both alleles with >=20 CA repeats (SEQ ID NO: 7)) for EGFR-CA-repeat in intron 1;
(b) (C/C or C/T) for MTHFR C677T; or
(c) (C/C or A/C) for MTHFR A12980,
comprising administering to the patient an effective amount of the therapy, thereby treating the patient.

8.-9. (canceled)

10. The method of claim 7, wherein the patient was selected by a method comprising determining a genotype of a cell or tissue sample isolated from the patient for at least one polymorphism of the group EGFR-CA-repeat in intron 1, MTHFR C677T, or MTHFR A1298C.

11. A method for identifying a cancer patient that is likely to experience a longer or shorter progression free survival following a therapy comprising a topoisomerase inhibitor, comprising determining a genotype of a cell or tissue sample isolated from the patient for an EGFR-CA-repeat in intron 1 polymorphism, wherein a genotype of (both alleles with >=20 CA repeats (SEQ ID NO: 7)) for EGFR-CA-repeat in intron 1 identifies the patient as likely to experience a longer progression free survival, or a genotype of (at least one allele with <20 CA repeats (SEQ ID NO: 8)) for EGFR-CA-repeat in intron 1 identifies the patient as likely to experience a shorter progression free survival.

12. The method of claim 11, wherein a genotype of (both alleles with >=20 CA repeats (SEQ ID NO: 7)) for EGFR-CA-repeat in intron 1 identifies the patient as likely to experience a longer progression free survival.

13. The method of claim 11, wherein a genotype of (at least one allele with <20 CA repeats (SEQ ID NO: 8)) for EGFR-CA-repeat in intron 1 identifies the patient as likely to experience a shorter progression free survival.

14. A method for identifying a cancer patient that is likely to experience a longer or shorter overall free survival following a therapy comprising a topoisomerase inhibitor, comprising determining a genotype of a cell or tissue sample isolated from the patient at least one polymorphism of the group MTHFR C677T or MTHFR A1298C, wherein a genotype of one or more of:
(a) (C/C or C/T) for MTHFR C677T; or
(b) (C/C or A/C) for MTHFR A1298C
identifies the patient as likely to experience a longer overall survival, or a genotype of neither (a) nor (b) identifies the patient as likely to experience a shorter overall survival.

15. The method of claim 14, wherein a genotype of one or more of:
(a) (C/C or C/T) for MTHFR C677T; or
(b) (C/C or A/C) for MTHFR A1298C
identifies the patient as likely to experience a longer overall survival.

16. The method of claim 14, wherein a genotype of neither (a) nor (b) identifies the patient as likely to experience a shorter overall survival.

17. A method for identifying a cancer patient that is more or less likely to respond a therapy comprising a topoisomerase inhibitor, comprising determining a genotype of a cell or tissue sample isolated from the patient for a MTHFR A1298C polymorphism, wherein a genotype of (C/C or A/A) for MTHFR A1298C identifies the patient as more likely to respond to the therapy, or a genotype of (A/C) for MTHFR A1298C identifies the patient as less likely to respond to the therapy.

18. The method of claim 17, wherein a genotype of (C/C or A/A) for MTHFR A1298C identifies the patient as more likely to respond to the therapy.

19. The method of claim 17, wherein a genotype of (A/C) for MTHFR A1298C identifies the patient as less likely to respond to the therapy.

20. A method for treating a cancer patient selected for a therapy comprising a topoisomerase inhibitor as likely to respond to the therapy based on the presence of a (C/C or A/A) for MTHFR A1298C genotype, comprising administering to the patient an effective amount of the therapy, thereby treating the patient.

21.-22. (canceled)

23. The method of claim 20, wherein the patient was selected by a method comprising determining a genotype of a cell or tissue sample isolated from the patient for a MTHFR A1298C polymorphism.

24. The method of claim 1, wherein the therapy comprises administration of irinotecan or an equivalent thereof.

25. The method of claim 1, wherein the therapy comprises administration of a topoisomerase inhibitor in combination with an anti-EGFR drug.

26. The method of claim 25, wherein the anti-EGFR drug is cetuximab or an equivalent thereof.

27. The method of claim 25, wherein the administration of the topoisomerase inhibitor and the anti-EGFR drug is concurrent or sequential.

28. The method of claim 1, wherein the topoisomerase inhibitor therapy is a second line therapy.

29. The method of claim 1, wherein the cancer patient is suffering from at least one cancer of the group colorectal cancer or head and neck cancer.

30. The method of claim 29, wherein the colorectal cancer is metastatic colorectal cancer.

31. The method of claim 1, wherein the sample comprises at least one of a tumor cell, a normal cell adjacent to a tumor, a normal cell corresponding to the tumor tissue type, a blood cell, a peripheral blood lymphocyte, or combinations thereof.

32. The method of claim 1, wherein the sample is at least one of a fixed tissue, a frozen tissue, a biopsy tissue, a resection tissue, a microdissected tissue, or combinations thereof.

33. The method of claim 1, wherein the determination is by a method comprising PCR, PCR-RFLP, sequencing, or microarray.

34. The method of claim 1, wherein the patient is an animal patient.

35. The method of claim 34, wherein the animal patient is a mammalian, simian, murine, bovine, equine, porcine or ovine patient.

36. The method of claim 35, wherein the patient is a human patient.

37.-48. (canceled)


SEQUENCE LISTING (9 sequences)

SEQ ID NO: 1 - DNA, 20 bp, Artificial Sequence
  Description of Artificial Sequence Synthetic primer
accccagggctctatgggaa

SEQ ID NO: 2 - DNA, 20 bp, Artificial Sequence
  Description of Artificial Sequence Synthetic primer
tgagggcacaagaagcccct

SEQ ID NO: 3 - DNA, 23 bp, Artificial Sequence
  Description of Artificial Sequence Synthetic primer
tgaaggagaaggtgtctgcggga

SEQ ID NO: 4 - DNA, 20 bp, Artificial Sequence
  Description of Artificial Sequence Synthetic primer
aggacggtgcggtgagagtg

SEQ ID NO: 5 - DNA, 25 bp, Artificial Sequence
  Description of Artificial Sequence Synthetic primer
ctttggggagctgaaggactactac

SEQ ID NO: 6 - DNA, 23 bp, Artificial Sequence
  Description of Artificial Sequence Synthetic primer
cactttgtgaccattccggtttg

SEQ ID NO: 7 - DNA, 40 bp, Artificial Sequence
  Description of Artificial Sequence Synthetic oligonucleotide
cacacacacacacacacacacacacacacacacacacaca

SEQ ID NO: 8 - DNA, 38 bp, Artificial Sequence
  Description of Artificial Sequence Synthetic oligonucleotide
cacacacacacacacacacacacacacacacacacaca

SEQ ID NO: 9 - DNA, 52 bp, Artificial Sequence
  Description of Artificial Sequence Synthetic oligonucleotide
cacacacacacacacacacacacacacacacacacacacacacacacacaca